(19) United States
(12) Patent Application Publication
(10) Pub. No.: US 2012/0178824 A1
(43) Pub. Date: Jul. 12, 2012

(54) HYDROGELS BASED ON ESTERS OF POLYISOBUTENESUCCINIC ACID

(75) Inventors: Hannah Maria König, Mannheim (DE); Sophia Ebert, Mannheim (DE); Roland Ettl, Altussheim (DE); Ouidad Benlahmar, Mannheim (DE); Marc-Steffen Schiedel, Dusseldorf (DE); Brigitte Giesen, Dusseldorf (DE); Petra Plantikow, Dusseldorf (DE)

(73) Assignees: Henkel AG & Co. KGaA, Dusseldorf (DE); BASF SE, Ludwigshafen (DE)

(21) Appl. No.: 13/347,255

(22) Filed: Jan. 10, 2012

Related U.S. Application Data
(60) Provisional application No. 61/431,450, filed on Jan. 11, 2011.

(51) Int. Cl. A01N 25/24; A61K 8/85; C08L 23/26; A01P 1/00; C08F 10/10; C11D 17/00; A61K 47/34

(52) U.S. Cl. 514/772.1; 512/4; 510/109; 510/403; 525/333.7; 524/576

(57) ABSTRACT

The present invention relates to the use of esters of polyisobutenesuccinic acid for producing hydrogels, and to the use of such hydrogels for cleaners and care compositions for the home (so-called homecare products), for cosmetics, and also for medical products.

The present invention relates to the use of esters of polyisobutenesuccinic acid for producing hydrogels, and to the use of such hydrogels for cleaners and care compositions for the home (so-called homecare products), for cosmetics, and also for medical products.

Hydrogels, i.e. water-comprising gels based on crosslinked, water-swellable but simultaneously water-insoluble polymers are of interest for a very wide variety of applications. Depending on the type of polymer, they are used as biomaterials in the pharmaceutical or medical sector, for example for contact lenses, wound closure materials, soft implants, for coating surfaces, for example biomedical articles such as implants or contact lenses, for producing biosensors (see Römpp Chemie-Lexikon, 10th edition, Georg Thieme Verlag 1997, p. 1835 and literature cited therein). Hydrogels laden with perfume or surfactants are sometimes used in fragrance dispensers or as cleaners. Despite a large number of known synthetic and natural polymeric hydrogel formers such as poly(meth)acrylic acids, polyvinyl alcohols, polyvinylpyrrolidones, polyalkylene ethers, pectins, alginates and the like, there is a continuous need for new gel formers.

EP 1318191 discloses water-containing pastes for fragrance release for the sanitary sector which, besides water and perfume substances, comprise a block copolymer which has oligo- or polyethylene oxide, oligo- or polypropylene oxide, or oligo- or polybutylene oxide groups. Specifically, polyoxyethylene-polyoxypropylene di- and triblock copolymers are specified. Pastes of this type adhere well to ceramic surfaces and are not rinsed off as a whole under the action of water, but dissolve slowly and completely only after or during frequently repeated action of water. It has proven disadvantageous that, in the event of relatively infrequent action of water and/or in the event of prolonged intervals between repeated actions of water, pastes of this type have a tendency to dry out and can then no longer be removed completely. These dried-out pastes look unpleasant too. A further disadvantage of these pastes is their low dimensional stability, as a result of which they run down the ceramic wall and form unattractive “noses”.

The object of the present invention is to provide new gel formers for hydrogels. These gel formers should form hydrogels which are dimensionally stable at least over a prolonged period, and moreover have no or no significant surface-active properties. Furthermore, biocompatibility is desirable.

WO 02/02674 describe block copolymers, in particular triblock and higher multiblock copolymers, which are obtainable by reacting silane-terminated polyisobutene with allyl-terminated polyalkylene glycol ethers. The block copolymers are swellable with water. Their production is comparatively complex. The properties of the hydrogels produced therefrom, especially their mechanical properties, are unsatisfactory.

DE 10125158 describes, inter alia, esters of polyisobutenesuccinic acid with an alcohol selected from poly-C₂-C₄-alkylene glycols and the use thereof as emulsifier for water-in-oil emulsions.

WO 2007/014915 describes aqueous polymer dispersions of polyolefins using polyisobutenes functionalized with hydrophilic groups, such as, for example, esters of polyisobutenesuccinic acid with an alcohol selected from poly-C₂-C₄-alkylene glycols, as emulsifiers.

The use of such esters for producing hydrogels has hitherto not been described.

Surprisingly, it has been found that esters of polyisobutenesuccinic acid with an alcohol selected from poly-C₂-C₄-alkylene glycols and poly-C₂-C₄-alkylene glycol mono-C₁-C₂₂-alkyl ethers form stable hydrogels with water, i.e. act as gel formers.

The invention therefore relates to the use of esters of polyisobutenesuccinic acid with an alcohol selected from poly-C₂-C₄-alkylene glycols and poly-C₂-C₄-alkylene glycol mono-C₁-C₂₂-alkyl ethers in hydrogels or as gel former for hydrogels, in particular in hydrogels which can be used in cleaners and care compositions for the home (homecare products), in cosmetics, and also for medical products.

The invention also relates to hydrogels, in particular hydrogels for cleaners and care compositions for the home, for cosmetics, and also for medical products, where the hydrogels comprise, besides water, at least one ester of polyisobutenesuccinic acid with an alcohol selected from poly-C₂-C₄-alkylene glycols and poly-C₂-C₄-alkylene glycol mono-C₁-C₂₂-alkyl ethers.

The invention also relates to the use of esters of this type for producing hydrogels, and to a process for producing the hydrogels, in which at least one ester of polyisobutenesuccinic acid with an alcohol selected from poly-C₂-C₄-alkylene glycols and poly-C₂-C₄-alkylene glycol mono-C₁-C₂₂-alkyl ethers is incorporated into an aqueous liquid, or mixed with the aqueous liquid.

The hydrogels according to the invention are stable, i.e. they are dimensionally stable over a large temperature range from, for example, 0 to 90° C., in particular 0 to 70° C., and do not have a tendency to separate even upon mechanical stress. The gel formers present therein, i.e. the esters of polyisobutenesuccinic acid described here, moreover, do not exhibit surface-active properties, i.e. at a concentration of 1 g/l, they do not lower the surface tension of the water below 45 mN/m, determined by the ring method in accordance with DIN 53914: 1980-03 at 25° C. On account of the gel formers used, the hydrogels are, moreover, biocompatible, i.e. they have no, or no noteworthy, disadvantageous effect on living beings or living material such as cell material or tissue.

The hydrogels according to the invention have good adhesion on polar surfaces, in particular inorganic surfaces such as glass or ceramic, and are not immediately rinsed off upon the action of water, but dissolve without leaving a residue, only after prolonged and frequently repeated action of water. Moreover, they can be formulated without disadvantages with fragrances or other substances which promote the cleaning or disinfection of sanitary ceramicware. In addition, these hydrogels only have a slight tendency to dry out. Furthermore, the hydrogels are dimensionally stable and are therefore suitable for producing molded articles, e.g. in fragrance dispensers.

The hydrogels according to the invention can be easily formulated with fragrances or other additives for cleaners, such as, for example, surfactants, dyes, preservatives, disinfectants, complexing agents, thickeners, humectants, disintegrants, foam stabilizers or substances which dissolve lime or urine scale, and are especially suitable for use in the sanitary sector. They adhere well to ceramic surfaces and are not rinsed off as a whole under the action of water, but dissolve slowly and completely only after frequently repeated action of water. In particular, it has proven advantageous that, in the event of infrequent action of water and/or in the case of prolonged intervals between the repeated actions of water, pastes of this type have no, or only a low, tendency to dry out and can be removed completely by repeated rinsing with water even in cases of relatively infrequent action of water.

A hydrogel former is understood as meaning a polymer which forms stable hydrogels with water upon the action of water and the associated swelling at least within a certain temperature range, e.g. in the range from 5 to 40° C. A stable hydrogel is understood as meaning a hydrogel which does not separate in a significant way upon mechanical stress and/or prolonged storage, at least within a certain temperature range, e.g. in the range from 5 to 40° C., i.e. at which no significant deposition of an aqueous serum takes place under these conditions.

Without being bound to one theory, it is assumed that in the hydrogels according to the invention the ester of polyisobutenesuccinic acid binds the water to form a 3-dimensional, polymeric network, with the polyalkylene groups of the ester presumably bringing about the binding of the water and the good adhesion to the polar surfaces, whereas the nonpolar polyisobutenyl radicals, on account of hydrophobic interactions and association, lead to a physical, i.e. non-covalent, crosslinking of the polymer chains and thus to the formation of a three-dimensional, dimensionally stable polymer network.

Polyisobutenesuccinic acid is understood as meaning oligomeric or polymeric macromolecules with an oligomer radical or polymer radical, respectively, which is derived from isobutene and which has, on one of its termini 1 or 2, radicals derived from succinic acid, i.e. radicals of the formula SA

—CH(COOH)CH₂COOH (SA)

and accordingly 2 or 4 carboxyl groups, and also mixtures thereof.

Polyisobutenesuccinic acids can therefore be described by the following formulae IIa and IIb:

PIB—CH(COOH)CH₂COOH (IIa)

PIB′—[CH(COOH)CH₂COOH]₂ (IIb)

where PIB in formula IIa is a monovalent oligomer radical or polymer radical derived from polyisobutene, and PIB′ in formula IIb is a divalent oligomer radical or polymer radical derived from polyisobutene.

In the esters of polyisobutenesuccinic acid used according to the invention, at least one of the carboxyl groups is present in the form of the ester with a poly-C₂-C₄-alkylene glycol or a poly-C₂-C₄-alkylene glycol mono-C₁-C₂₂-alkyl ether. Esters of this type can be described by the general formulae Ia and Ib:

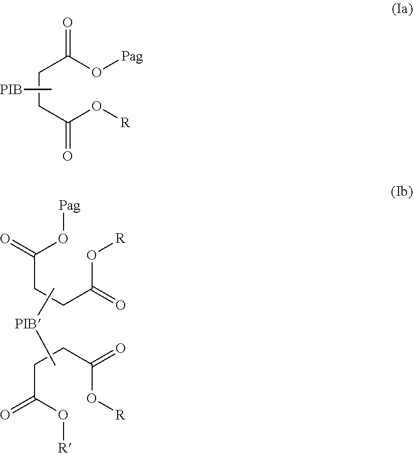

in which PIB and PIB′ have the meanings given above for formulae IIa and IIb, R and R′, independently of one another, are hydrogen or Pag and Pag is a radical derived from a poly-C₂-C₄-alkylene glycol or a poly-C₂-C₄-alkylene glycol mono-C₁-C₂₂-alkyl ether. In the formulae Ia and Ib, R is in particular hydrogen.

Poly-C₂-C₄-alkylene glycols are understood as meaning linear or branched oligomers or polymers which are composed essentially of repeat units of the formula -A-O— (hereinbelow also alkylene oxide repeat units), in which A is C₂-C₄-alkanediyl, and which have hydroxyl groups on their termini.

Poly-C₂-C₄-alkylene glycol mono-C₁-C₂₂-alkyl ethers are understood as meaning linear or branched oligomers or polymers which are composed essentially of repeat units of the formula -A-O—, in which A is C₂-C₄-alkanediyl, which have, at one of their ends, a C₁-C₂₂-alkyl group bonded via oxygen, and which have hydroxyl groups at the other terminus or the other termini.

In these poly-C₂-C₄-alkylene glycols and poly-C₂-C₄-alkylene glycol mono-C₁-C₂₂-alkyl ethers, the repeat units of the formula -A-O— may be identical or different. If the poly-C₂-C₄-alkylene glycols and poly-C₂-C₄-alkylene glycol mono-C₁-C₂₂-alkyl ethers have different repeat units of the formula -A-O—, these may be arranged randomly, alternately or in a plurality, e.g. 2, 3 or 4, blocks. In one specific embodiment of the invention, the from the poly-C₂-C₄-alkylene glycols and poly-C₂-C₄-alkylene glycol mono-C₁-C₂₂-alkyl ethers have different repeat units of the formula -A-O— which are arranged randomly.

In this context, C₂-C₄-alkanediyl is a saturated divalent hydrocarbon radical having 2 to 4 carbon atoms, such as 1,2-ethanediyl, 1,2-propanediyl, 1,3-propanediyl, 1,4-butanediyl, 1,2-butanediyl, 1,3-butanediyl, 2,3-butanediyl or 1-methyl-1,2-propanediyl.

In this context, C₁-C₂₂-alkyl is a saturated, acyclic monovalent hydrocarbon radical having 1 to 22 carbon atoms, in particular having 1 to 8 carbon atoms or 1 to 4 carbon atoms, such as methyl, ethyl, n-propyl, isopropyl, 1-butyl, 2-butyl, tert-butyl, isobutyl, n-pentyl, isopentyl, n-hexyl, isohexyl, n-heptyl, isoheptyl, n-octyl, isooctyl, 2-ethylhexyl, n-nonyl, isononyl, n-decyl, 2-propylheptyl, n-undecyl, n-dodecyl, n-tridecyl, myristyl, pentadecyl, palmityl (=cetyl), heptadecyl, octadecyl, nonadecyl, arachinyl or behenyl.

Polymer radicals derived from isobutene, hereinbelow also polyisobutenyl radicals, are understood as meaning organic radicals which are derived from linear or branched oligomers or polymers of isobutene and which can comprise, polymerized therein, up to 20% by weight, preferably not more then 10% by weight, of C₂-C₁₂-olefins different from isobutene, such as 1-butene, 2-butene, 2-methyl-1-butene, 2-methylpentene-1, 2-methylhexene-1,2-ethylpentene-1,2-ethylhexene-1,2-propylheptene-1. Radicals of this type can be described in the case of monovalent radicals PIB for example by the following formulae

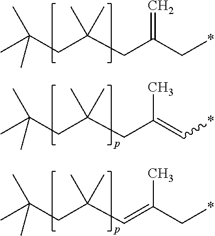

and in the case of divalent radicals PIB′, for example by the following formulae

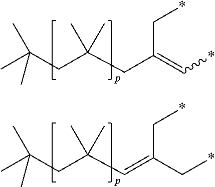

in which the value p+2 corresponds to the degree of polymerization and indicates the number of isobutene units in the polyisobutene radical and * signifies the linkage to the succinic acid (ester) radical. In these formulae, some of the isobutene units —CH₂C(CH₃)₂—, generally not more than 20% by weight, preferably not more than 10% by weight, can be replaced by C₂-C₁₂-alkane-1,2-diyl groups derived from C₂-C₁₂-olefins which are different therefrom. The degree of polymerization p+2 is typically in the range from 5 to 100, in particular in the range from 8 to 80 and specifically in the range from 15 to 65.

With regard to the use according to the invention in hydrogels, preference is given to those esters of polyisobutenesuccinic acid which, based on the total weight of the ester, consist to at least 50% by weight, in particular to at least 70% by weight, of esters of the formula Ia. Preferably, the esters of polyisobutenesuccinic acid comprise, based on the total weight of the ester, less than 30% by weight, in particular less than 20% by weight, of esters of the formula Ib.

As a consequence of the preparation, the esters of polyisobutenesuccinic acid may comprise unmodified polyisobutene. Unless stated otherwise, this is not included in the esters here and below. The fraction of polyisobutene can constitute up to 50% by weight, but preferably not more than 40% by weight or not more than 30% by weight, based on the total amount of ester+polyisobutene.

With regard to the use according to the invention in hydrogels, preference is given to those esters of polyisobutenesuccinic acid whose polyisobutene radical of the ester has a number-average molecular weight in the range from 500 to 5000 daltons, in particular in the range from 800 to 3600.

In a specific embodiment of the invention, polyisobutene radicals of the polyisobutenesuccinic acid esters have a narrow molecular weight distribution. The polydispersity is then preferably at most 1.4, particularly preferably at most 1.3, in particular at most 1.2. Polydispersity is understood as meaning the quotient of weight-average molecular weight M_{w} and number-average molecular weight Mₙ (PDI=M_{w}/Mₙ).

With regard to the use according to the invention in hydrogels, preference is given to those esters of polyisobutenesuccinic acid which are esterified with an alcohol selected from poly-C₂-C₄-alkylene glycols poly-C₂-C₄-alkylene glycol mono-C₁-C₂₂-alkyl ethers, or a mixture of these alcohols, where the alcohol or the alcohols has or have a number-average molecular weight in the range from 500 to 15 000 daltons, in particular in the range from 800 to 10 000 daltons and specifically in the range from 1200 to 5000 daltons.

Furthermore, it has proven to be advantageous if the alcohol which is esterified with the polyisobutenesuccinic acid is unbranched, i.e. is selected from linear poly-C₂-C₄-alkylene glycols and linear poly-C₂-C₄-alkylene glycol mono-C₁-C₂₀-alkyl ethers. Unbranched, i.e. linear poly-C₂-C₄-alkylene glycols and linear poly-C₂-C₄-alkylene glycol mono-C₁-C₂₀-alkyl ethers can be described by the following formula (III):

HOA-OₙR′ (III)

Here, A is C₂-C₄alkanediylas defined above, which may be identical or different and which is preferably selected from 1,2-ethanediyl and 1,2-propanediyl. R′ is hydrogen or C₁-C₂₂-alkyl, in particular hydrogen or C₁-C₁₀-alkyl and specifically hydrogen or C₁-C₄-alkyl, e.g. methyl. The variable n indicates the average number of repeat units [A-O] (number-average) and is typically in the range from 10 to 350, in particular in the range from 15 to 200.

Accordingly, the radical Pag in the formulae Ia and Ib is preferably a radical of the formula

*A-OₙR′ (Pag)

in which A, R and n have the meanings given above and * signifies the linkage to the oxygen atom of the polyisobutenesuccinic acid radical.

In formula III or in formula Pag, the repeat units of the formula -A-O— may be identical or different. If the formulae III or in formulae Pag have different repeat units of the formula -A-O—, these may be arranged randomly or in a plurality, e.g. 2, 3 or 4 blocks. In a specific embodiment of the invention, the formulae III and in formulae Pag have different repeat units of the formula -A-O—, which are arranged randomly.

Furthermore, it has proven to be advantageous if the alcohol which is esterified with the polyisobutenesuccinic acid is composed, to at least 50 mol %, and in particular to at least 70 mol %, based on the total number of alkylene oxide repeat units in the alcohol, of repeat units of the formula [CH₂CH₂O]. Accordingly, in the formulae III and Pag, the fraction of repeat units of the formula [CH₂CH₂O] is at least 50 mol %, and in particular at least 70 mol %, based on the total number of repeat units A-O.

In a specific embodiment of the invention, all or virtually all of the repeat units A-O of the poly-C₂-C₄-alkylene glycols or of the poly-C₂-C₄-alkylene glycol mono-C₁-C₂₀-alkyl ether, or all or virtually all of the repeat units A-O in the formulae III and Pag, are repeat units of the formula [CH₂CH₂O].

In a further preferred embodiment of the invention, the alcohol which is esterified with the polyisobutenesuccinic acid, in particular the alcohol of the formula III or the radical Pag, comprises
50 mol % to 99 mol %, and in particular 70 mol % to 98 mol %, based on the total number of alkylene oxide repeat units in the alcohol, of repeat units of the formula [CH₂CH₂O], and
1 mol % to 50 mol %, and in particular 2 mol % to 30 mol %, based on the total number of alkylene oxide repeat units in the alcohol, of repeat units of the formula [A′-0], in which A′ is C₃-C₄-alkanediyl, and in particular repeat units of the formula [CH₂CH(CH₃)O].

In one specific version of this preferred embodiment, the repeat units [CH₂CH₂O] and [A′-O] which are different from one another are not arranged in a blocklike manner, but are in random distribution or arranged alternately.

In addition, it has proven to be advantageous if the alcohol constituent and the polyisobutenesuccinic acid on which the ester is based is selected such that the ester has, on average, a weight ratio of polyisobutene radical to alcohol radical in the range from 10:1 to 1:30, preferably in the range from 1.5:1 to 1:20 and in particular in the range from 1:1 to 1:10 to.

The preparation of the esters of polyisobutene succinic acid used according to the invention is possible in a manner known per se by reacting polyisobutenesuccinic acid or an ester-forming derivative of polyisobutenesuccinic acid with a poly-C₂-C₄-alkylene glycol or poly-C₂-C₄-alkylene glycol mono-C₁-C₂₂-alkyl ether or mixtures thereof and esterification conditions. Processes for this are known in principle, e.g. from DE 10125158 and WO 2007/014915 cited at the start.

Suitable ester-forming derivatives of polyisobutenesuccinic acid are the acid halides and the C₁-C₄-alkyl esters of polyisobutenesuccinic acid and also as in particular polyisobutenesuccinic anhydride.

In one preferred embodiment of the invention, ester of polyisobutenesuccinic acid is used which is obtainable by reacting polyisobutenesuccinic anhydride with an alcohol selected from poly-C₂-C₄-alkylene glycols and poly-C₂-C₄-alkylene glycol mono-C₂-C₂₀-alkyl ethers, in particular an alcohol of the formula III, or a mixture of these alcohols.

Polyisobutenesuccinic anhydride is understood here and below as meaning the internal anhydrides of polyisobutenesuccinic acid, i.e. substances in which the two carboxyl groups of the succinic acid radical form a 1-oxolane-2,5-dion-2-yl radical. Polyisobutenesuccinic anhydride of this type can be described in particular by the following formulae

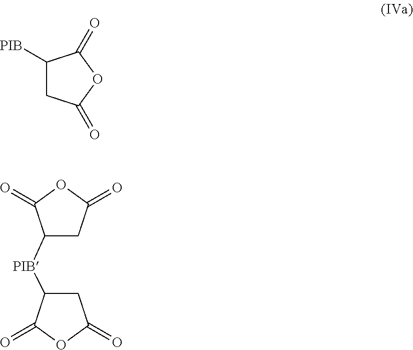

in which PIB and PIB′ have the meanings given above for formulae Ia, Ib, IIa and IIb.

Preferably, the polyisobutenesuccinic anhydride used for producing the ester comprises, based on the total weight of the anhydride, to at least 50% by weight, in particular to at least 70% by weight, the anhydride of formula IVa. Preferably, the polyisobutenesuccinic anhydride used for producing the ester comprises, based on the total weight of the anhydride, less than 30% by weight, in particular less than 20% by weight, of anhydride of the formula IVb. As a consequence of the preparation, the polyisobutenesuccinic anhydride can comprise polyisobutene. The fraction of the polyisobutene can constitute up to 50% by weight, but preferably not more than 40% by weight or not more than 30% by weight, based on the total amount of polyisobutenesuccinic anhydride+polyisobutene.

The relative fraction of compounds of the formula IVa and IVb in the polyisobutenesuccinic anhydride used to produce the ester corresponds to the saponification number of the polyisobutenesuccinic anhydride, determined analogously to DIN 53401. For the properties of the ester, it has proven to be advantageous if the polyisobutenesuccinic anhydride has a saponification number SN in the range from 40 to 140 mg KOH/g and in particular in the range from 70 to 100 mg KOH/g, determined in accordance with DIN 53401.

The polyisobutenesuccinic anhydrides used for the reaction are known, e.g. from DE 2702604 A1, U.S. Pat. No. 5,883,196, U.S. Pat. No. 5,420,207 and EP 629638, and also the publication by M. Tessier et al., Eur. Polym. J, 20, 1984, p. 269-280 and H. Mach et al., Lubrication Science 12-2, 1999, p. 175-185.

Preference is given to polyisobutenesuccinic anhydrides which are obtainable by reacting olefinically unsaturated polyisobutenes with maleic anhydride. Particular preference is given to products which are obtained by reacting highly reactive polyisobutenes with maleic anhydride. Highly reactive polyisobutenes are understood as meaning polyisobutenes with at least 50 mol %, often with at least 60 mol % and in particular with at least 80 mol %, based on the total number of polyisobutene macromolecules, of terminally arranged double bonds. The terminally arranged double bonds may either be vinyl double bonds [—CH═C(CH₃)₂] (β-olefin) or vinylidene double bonds [—CH—C(═CH₂)—CH₃] (α-olefin). Preferred highly reactive polyisobutene have predominantly vinylidene double bonds. Highly reactive polyisobutenes are commercially available, e.g. the Glissopal grades from BASF SE, thus e.g. Glissopal® 1000 and Glissopal® 1300, Glissopal® 2300.

The poly-C₂-C₄-alkylene glycols and poly-C₂-C₄-alkylene glycol mono-C₂-C₂₀-alkyl ethers used for the reaction are likewise known from the prior art and commercially available, for example under the trade names Pluriol®, e.g. the Pluriol® E grades such as Pluriol® E 600, Pluriol® E 600 S, Pluriol® E 1000, Pluriol® E 1000 S, Pluriol® E 1500, Pluriol® E 3400, Pluriol® E 6000, Pluriol® E 8000, Pluriol® E 9000, the Pluriol®P grades such as Pluriol® E 600, Pluriol® E 900, Pluriol® E 2000, Pluriol® E 4000, the Pluriol® A grades such as Pluriol® A 1020 E, Pluriol® A 2000 E, Pluriol® A 3010 E, Pluriol®A 5010 E, Pluriol®A 1020 PE, Pluronic®, e.g. the Pluronic® PE grades such as Pluronic® PE 3100, Pluronic® PE 3500, Pluronic® PE 4300, Pluronic® PE 6100, Pluronic® PE 6120, Pluronic® PE 6200, Pluronic® PE 6400, Pluronic® PE 6800, Pluronic® PE 7400, Pluronic® PE 8100, Pluronic® PE 9200, Pluronic® PE 9400, Pluronic® PE 10100, Pluronic® PE 10300, Pluronic® PE 10400 and Pluronic® PE 10500, or can be prepared analogously to standard processes by base-catalyzed homo- or copolymerization of C₂-C₄-alkylene oxides such as ethylene oxide, propylene oxide, 1,2-butylene oxide, 2-methyl-1,2-propylene oxide (=isobutylene oxide).

The reaction of the polyisobutenesuccinic anhydride with the alcohol selected from poly-C₂-C₄-alkylene glycols and poly-C₂-C₄-alkylene glycol mono-C₂-C₂₀-alkyl ethers can take place in a manner known per se analogously to the procedures described in DE 10125158 and WO 2007/014915.

For this, as a rule, the polyisobutenesuccinic anhydride is reacted with the alcohol selected from poly-C₂-C₄-alkylene glycols and poly-C₂-C₄-alkylene glycol mono-C₂-C₂₀-alkyl ethers in a molar ratio of 2:1 to 1:2, in particular 1.5:1 to 1:1.5 and specifically 1.05:1 to 1:1.2, in each case based on the anhydride groups in the polyisobutenesuccinic anhydride.

The reaction can be carried out in solution or without dilution. Examples of suitable solvents are aromatic hydrocarbons, e.g. benzene, toluene, xylenes, mesitylene, naphthalene, tert-butylbenzene, and mixtures thereof, (cyclo)aliphatic hydrocarbons, e.g. hexane, heptane, octane, isooctane, cyclohexane, cycloheptane, cyclooctane, tetralin, and mixtures thereof, halogenated hydrocarbons such as dichloromethane, 1,1-dichloroethane, 1,2-dichloroethane, 1,1-dichloroethene, 1,2-dichloroethene, chlorobenzene, dichlorobenzene, chlorotoluene and mixtures thereof, and also mixtures of the aforementioned aromatic and (cyclo)aliphatic hydrocarbons and mixtures of the aforementioned hydrocarbons with halogenated hydrocarbons.

The reaction can take place in the presence of a catalyst or in the absence of catalysts. As a rule, the reaction takes place at temperatures in the range from 60 to 250° C., often in the range from 80 to 200° C. and in particular in the range from 100 to 180° C. Suitable catalysts are in particular basic compounds such as alkali metal and alkaline earth metal oxides, hydroxides, carbonates and hydrogencarbonates, and also tertiary organic amines, e.g. trialkylamines such as triethylamine, tripropylamine, methyldiisopropylamine, tributylamine, dimethyl-tert-butylamine, and also cyclic alkylamines such as N-methylmorpholine, N-methylpiperidine, N-methylpyrrolidine, and triethylenediamine. If required, the catalyst is used in amounts of from 0.1 to 20 mol %, based on the anhydride groups in the polyisobutenesuccinic anhydride.

As already mentioned in the introduction, the esters of polyisobutenesuccinic acid with an alcohol selected from poly-C₂-C₄-alkylene glycols and poly-C₂-C₄-alkylene glycol mono-C₁-C₂₂-alkyl ethers form stable hydrogels with water, i.e. they can be used as gel formers.

Accordingly, the present invention also relates to hydrogels which, besides water (hereinbelow also component B), comprise at least one ester of polyisobutenesuccinic acid with an alcohol selected from poly-C₂-C₄-alkylene glycols and poly-C₂-C₄-alkylene glycol mono-C₁-C₂₂-alkyl ethers, as described above in an amount sufficient to form a hydrogel.

The amount of component A required to form the hydrogel naturally depends on the other constituents of the hydrogel and on the precise constitution of component A and can be ascertained easily by the person skilled in the art through routine experiments. As a rule, irrespective of the other additives, a stable hydrogel is obtained if the weight ratio of component A to component B, i.e. water, is in the range from 4:1 to 1:6, often in the range from 3:1 to 1:4 and in particular in the range from 2:1 to 1:3.

In the hydrogels according to the invention, the component A generally constitutes 15 to 80% by weight, often 20 to 75% by weight, and in particular 25 to 65% by weight, based on the total weight of the hydrogel.

In the hydrogels according to the invention, the total amount of components A and B is generally at least 70% by weight and in particular at least 80% by weight, of the hydrogel.

Typically, the hydrogel according to the invention comprises
a. 15 to 80% by weight, often 20 to 75% by weight, and in particular 25 to 65% by weight, based on the total weight of the hydrogel, of component A and
b. 20 to 85% by weight, often 25 to 80% by weight, and in particular 35 to 75% by weight, based on the total weight of the hydrogel, of water as component B.

Besides the aforementioned components, the hydrogel according to the invention can comprise one or more further constituents different from components A and B and which are directed to the desired intended use. These constituents are also referred below as component C.

Examples of component C are fragrances and customary additives present in cleaners, such as, for example, surfactants, dyes, preservatives, disinfectants, complexing agents, thickeners, humectants, disintegrants, foam stabilizers and substances which dissolve lime or urine scale, and mixtures of the aforementioned substances.

Accordingly, one embodiment of the invention relates to a hydrogel which comprises, besides component A and water (component B), at least one further constituent as component C, which is preferably selected from fragrances, surfactants, dyes, preservatives, disinfectants, complexing agents, thickeners, humectants, disintegrants, foam stabilizers and substances which dissolve lime or urine scale, and mixtures thereof.

The fraction of component C will generally not exceed 30% by weight, often 25% by weight and in particular 20% by weight, based on the total weight of the hydrogel, and is, if desired, typically in the range from 0.1 to 30% by weight and in particular in the range from 1 to 20% by weight.

The nature of component C is governed in a manner known per se by the desired intended use.

Accordingly, one embodiment of the invention relates to a hydrogel comprising:
a. 15 to 79.9% by weight, in particular 20 to 74.5% by weight, and specifically 25 to 64% by weight, based on the total weight of the hydrogel, of component A,
b. 20 to 84.9% by weight, in particular 25 to 79.5% by weight, and specifically 35 to 74% by weight, based on the total weight of the hydrogel, of water as component B,
c. 0.1 to 30% by weight, in particular 0.5 to 25% by weight, and specifically 1 to 20% by weight, based on the total weight of the hydrogel, of at least one further constituent different from components A and B, which is also referred to below as component C,
   where the total amount of components A, B and C is 100% by weight.

In one preferred embodiment of the invention, the hydrogel comprises at least one fragrance. Suitable fragrances which may be present in the hydrogels according to the invention comprise synthetic fragrances, semisynthetic fragrance mixtures and natural fragrance oils. Examples of synthetic fragrances are the synthetic products of the ester, ether, aldehyde, ketone, alcohol and hydrocarbon types. The natural fragrances include in particular those perfume oils which are accessible from plant sources. Preference is given to using mixtures of different fragrances which together produce a pleasant scent note.

In one preferred embodiment of the invention, the hydrogel according to the invention comprises at least one surfactant. Suitable surfactants are typically selected from anionic, nonionic, amphoteric and cationic surfactants, and also mixtures thereof. If desired, the hydrogels according to the invention comprise surfactants preferably in amounts of from 0.01 to 30% by weight, based on the total weight of the hydrogel. The hydrogels according to the invention can furthermore comprise one or more antimicrobial active ingredients, which can generally also act as preservative.

The hydrogels according to the invention can further comprise substances which dissolve lime or urine scale. These include in particular water-soluble builders and mixtures thereof with acids.

The hydrogels according to the invention can also comprise one or more conventional thickeners. Of suitability for this are in principle all viscosity regulators used in the prior art in detergents and cleaners. In one preferred embodiment of the invention, the hydrogel comprises no conventional thickener.

The hydrogels according to the invention are also largely dimensionally stable even under relatively large shear stresses, i.e. their deformability at 30° C. and a shear stress of 10² Pa is typically less than 5% and in particular less than 1%, determined at 30° C. using a shear-stress-controlled rotary viscometer with cone/plate geometry and a shear stress range from 10² to 10⁴ Pa. The yield point as a limit of the elastic deformation range is 30° C. as a rule at a shear stress of at least 10³ Pa, e.g. in the range from 10³ to 10⁶ Pa.

The hydrogels according to the invention typically have a viscosity in the range from 10⁵ to 10¹⁰ Pa·s, often in the range from 10⁵ to 10⁸ Pa·s, determined at 30° C. using a shear-stress-controlled rotary viscometer with cone/plate geometry in the shear stress range from 10² to 10⁴ Pa.

The hydrogels according to the invention have good adhesion on polar surfaces, in particular inorganic surfaces such as glass or ceramic, and are not immediately rinsed off upon action of water, but dissolve, without leaving a residue, only after prolonged and frequently repeated action of water. Moreover, they can be formulated without disadvantages with fragrances or other substances which promote the cleaning or disinfection of sanitary ceramicware. The invention therefore also relates to the use of a hydrogel as described here for homecare products, in particular for producing compositions which release fragrance, e.g. fragrance-releasing pastes or for producing cleaning and care compositions for the sanitary sector, specifically for pastes for application in WCs and bidets, as described in WO 99/66021, WO 02/26925 or EP 1318191.

The hydrogels according to the invention can be prepared in a simple manner by incorporating at least one ester of polyisobutenesuccinic acid with an alcohol selected from poly-C₂-C₄-alkylene glycols and poly-C₂-C₄-alkylene glycol mono-C₂-C₂₀-alkyl ethers, as described here, optionally with some or all of the constituents of component C into an aqueous liquid which, if desired, besides water, can already comprise some or the total amount of the constituents of component C.

The incorporation can be prepared by simply mixing water or an aqueous liquid which, besides water, comprises some or the total amount of the constituents of the optionally desired component C. However, it is also possible to incorporate a solution of component A, which optionally comprises some or all of the constituents of the optionally desired component C, into water or an aqueous liquid, and then to remove the solvent.

The incorporation of component A and optionally further constituents into water or the aqueous liquid will generally take place at temperatures in the range from 10 to 100° C. The use of mixing devices may be advantageous, but is generally not required.

The figures and examples below serve to illustrate the invention in more detail.

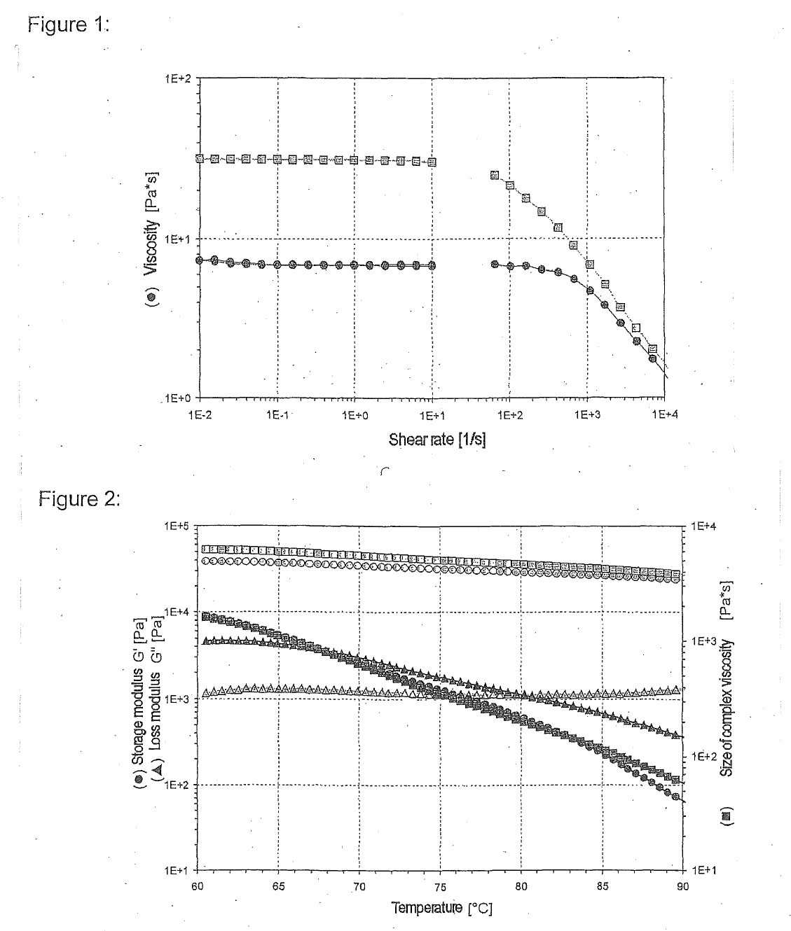

FIG. 1: Viscosity of the polyisobutenesuccinic acid ester from preparation example 11 as a function of the shear rate at 70° C. (grey) and 90° C. (black). Measuring instrument: stamping capillary viscometer.

FIG. 2: Temperature sweep of the polyisobutenesuccinic acid esters from preparation examples 11 (grey) and 12 (black) at temperatures of 60 to 90° C. where f=1 Hz and def.=0.1%, measuring instrument: shear-stress-controlled rotary rheometer.

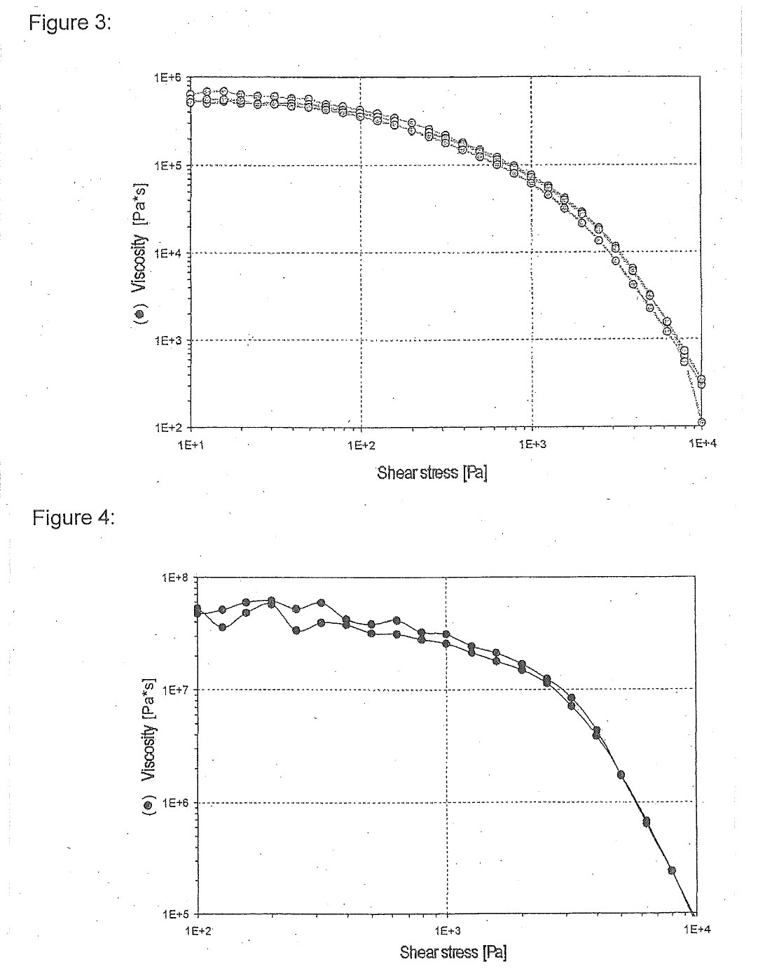

FIG. 3: Viscosities of the polyisobutenesuccinic acid esters from preparation example 11 as a function of shear stress, measuring instrument: shear-stress-controlled rotary viscometer.

FIG. 4: Viscosities of the hydrogel from example 21 as a function of shear stress; measuring instrument: shear-stress-controlled rotary viscometer.

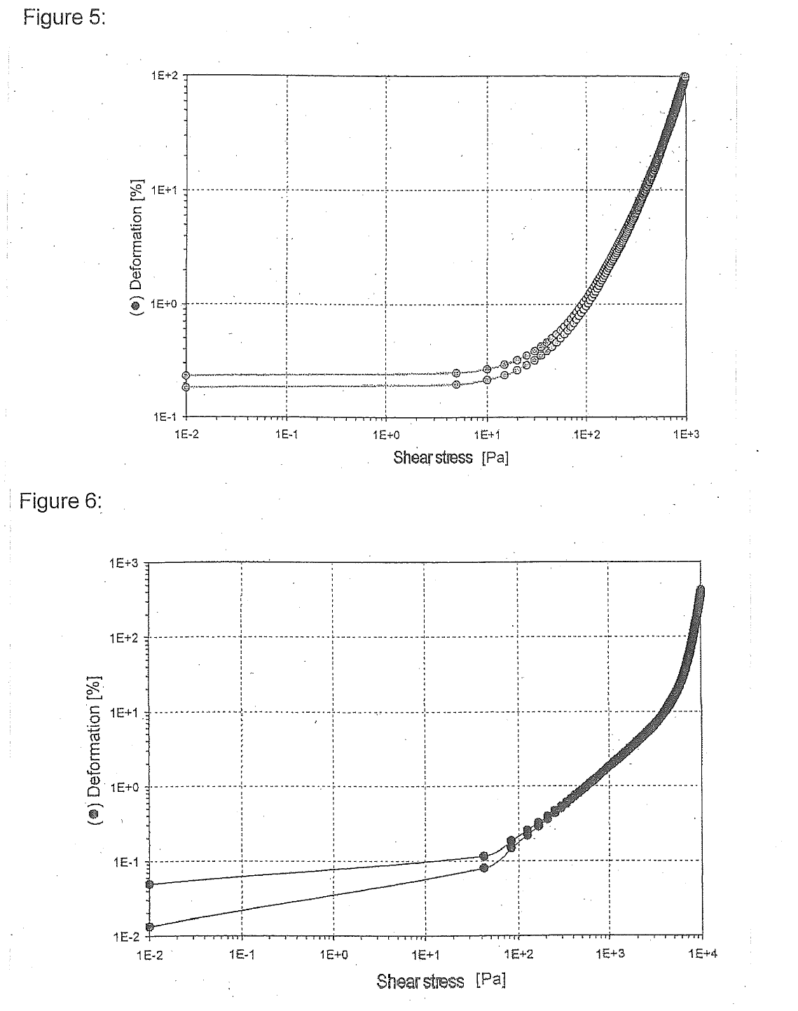

FIG. 5: Deformation of the polyisobutenesuccinic acid ester from preparation example 11 as a function of shear stress, measuring instrument: shear-stress-controlled rotary viscometer.

FIG. 6: Deformation of the hydrogel from example 21 as a function of shear stress, measuring instrument: shear-stress-controlled rotary viscometer.

### I ABBREVIATIONS

EO: ethylene oxide
PO: propylene oxide
PIBSA: polyisobutenesuccinic anhydride
Mₙ: number-average molecular weight
M_{w}: weight-average molecular weight
SN: saponification number
AN: acid number
OHN: OH number
ST: surface tension

### II ANALYTICS

The saponification number SN was determined analogously to DIN 53401:1998-06

The acid number AN was determined by titration of the polyisobutenesuccinic acid ester in a mixture of toluene and ethanol. The AN indicates the number of mg of potassium hydroxide which was used up to neutralize 1 g of the sample.

The OH number was determined analogously to DIN 53401:1971-12

The viscosity of the polyisobutenesuccinic acid esters was investigated by means of a shear-stress-controlled rotary rheometer (MCR300, plate/plate geometry, Ø 25 mm, h=1 mm) at temperatures of 60 to 90° C., and also by means of a stamping capillary viscometer (Rosand, KVM geometry, annular capillary: L/R=294.70, L=150.00 mm, R=0.509 mm) at temperatures of 70 and 90° C.

The deformability and the yield point of the polyisobutenesuccinic acid ester and the resulting hydrogel produced therefrom were determined by means of a shear-stress-controlled rotary viscometer (Physika MCR, plate/plate geometry, upper plate d 25 mm, distance: 2 mm) at a temperature of 30° C.

The surface tension ST was measured according to the ring method analogously to DIN 53914: 1980-03. The ST is defined as the force in the surface per unit of length and has the dimension mN/m (10⁻³ newtons/meter).

The maximum water absorption capacity of sample 11 was tested both with deionized water (demin. water) and also with non-deionized water (Jayco solution) both at room temperature and also at 4° C.

For this, ca. 3 g of sample were placed in a Petri dish and melted at 80° C. in a heating oven. After the sample had cooled back to room temperature, either demin. water or Jayco solution was added, a ratio of sample to water of 1:9 being established. The swelling behavior of sample 11 was then determined gravimetrically.

The Jayco solution comprised the following salt concentrations: 2 g/l potassium chloride, 2 g/l sodium sulfate, 0.85 g/l ammonium dihydrogenphosphate, 0.15 g/l diammonium hydrogenphosphate, 0.5 g/1 magnesium chloride hexahydrate, 0.25 g/l calcium chloride dihydrate.

### III FEED MATERIALS

Polyisobutenesuccinic anhydride 1: PIBSA with a saponification number SN of 87.5 mg KOH/g, prepared by reacting polyisobutene (Mₙ=1000 g/mol) with maleic anhydride (PIBSA 1000)

Polyisobutenesuccinic anhydride 2: PIBSA with a saponification number SN of 44 mg KOH/g, prepared by reacting polyisobutene (Mₙ=2300 g/mol) with maleic anhydride (PIBSA 2300)

Polyisobutenesuccinic anhydride 3: PIBSA with a saponification number SN of 84 mg KOH/g, prepared by reacting polyisobutene (Mₙ=1000 g/mol) with maleic anhydride

Polyisobutenesuccinic anhydride 4: PIBSA with a saponification number SN of 105 mg KOH/g, prepared by reacting polyisobutene (Mₙ=1000 g/mol) with maleic anhydride

Polyisobutenesuccinic anhydride 5: PIBSA with a saponification number SN of 145 mg KOH/g, prepared by reacting polyisobutene (Mₙ=550 g/mol) with maleic anhydride (PIBSA 550)

Polyether 1: random poly(ethylene glycol-co-propylene glycol)monomethyl ether (EO/PO ratio 10, Mₙ=2587 g/mol; SN=21.6 mg KOH/g)

Preparation of polyether 1: 69.7 g of diethylene glycol monomethyl ether and 3.1 g of an aqueous 50% strength by weight potassium hydroxide solution were introduced as initial charge in an autoclave. The mixture was heated to 80° C. and a vacuum of 10 mbar was applied for 2 h in order to remove the water. The system was then rendered inert with nitrogen and the reaction mixture was heated to 130° C. At this temperature, a mixture of 1277.8 g of ethylene oxide (EO) and 168.4 g of propylene oxide (PO) was injected over the course of 5 h and the mixture was after-stirred for 2 h at 130° C. The volatile constituents were then removed from the reaction mixture in vacuo, giving 1570 g of a white solid, which consisted essentially of KOH and the random EO/PO copolymer.

Polyether 2: random poly(ethylene glycol-co-propylene glycol)monomethyl ether (EO/PO ratio 20:3; Mₙ=1175 g/mol) prepared analogously to the preparation of polyether 1.

Polyether 3: random poly(ethylene glycol-co-propylene glycol)monomethyl ether (EO/PO ratio 50:3; Mₙ=2497 g/mol; SN=22.7 mg KOH/g), prepared analogously to the preparation of polyether 1.

Polyether 4: random poly(ethylene glycol-co-propylene glycol)monomethyl ether (EO/PO ratio 75:7.5; Mₙ=3860 g/mol; SN=16.8 mg KOH/g), prepared analogously to the preparation of polyether 1.

Polyether 5: random poly(ethylene glycol-co-propylene glycol)monomethyl ether (EO/PO ratio 10; Mₙ=5106 g/mol; SN=13.2 mg KOH/g), prepared analogously to the preparation of polyether 1.

Polyether 6: random poly(ethylene glycol-co-propylene glycol)monomethyl ether (EO/PO ratio 52:3; Mₙ=2623 g/mol; SN=23.9 mg KOH/g), prepared analogously to the preparation of polyether 1.

Polyether 7: polyethylene glycol, Mₙ=1500 g/mol

Polyether 8: polyethylene glycol, Mₙ=6000 g/mol

Polyether 9: polyethylene glycol monomethyl ether, Mₙ=2000 g/mol

Polyether 10: polyethylene glycol monomethyl ether, Mₙ=3010 g/mol

Polyether 11: polyethylene glycol monomethyl ether, Mₙ=5010 g/mol

Polyether 12: polyethylene glycol monomethyl ether, Mₙ=1020 g/mol

Polyether 13: poly(ethylene glycol-co-propylene glycol)monomethyl ether, Mₙ=1020 g/mol, molar ratio EO/PO 1:1

Polyether 14: polyethylene glycol, Mₙ=600 g/mol

Polyether 15: polyethylene glycol, Mₙ=1000 g/mol

Surfactant: nonionic surfactant

### IV PREPARATION EXAMPLES

### Preparation Example 1

### Polyisobutenesuccinic Acid Ester

Polyisobutenesuccinic anhydride 2 (0.0506 mol; 129 g) was reacted with polyether 7 (0.0506 mol; 75.9 g) at a temperature of 140° C. without dilution. The reaction time was 3 hours. The acid number of the copolymer obtained was 12.6 mg KOH/g.

### Preparation Examples 2 to 16

The polyisobutenesuccinic acid esters of preparation examples 2 to 16 were prepared in a manner analogous to preparation example 1. The feed materials, relative use amounts and the properties are summarized in table 1 below.

TABLE 1

| | | | PIBSA: | | |
|---|---|---|---|---|---|
| Preparation | PIBSA | Polyether | polyether | AN | ST |
| example No. | No. | No. | [mol:mol] | [mg KOH/g] | [mN/m] |
| 1 | 2 | 7 | 1:1 | 12.6 | |
| 2 | 1 | 8 | 1:1 | 7.9 | |
| 3 | 1 | 9 | 1:1 | 17.8 | 50.2 |
| 4 | 1 | 10 | 1:1 | 12.5 | 54.6 |
| 5 | 1 | 11 | 1:1 | 3.6 | |
| 6 | 1 | 12 | 1:1 | 8.8 | |
| 7 | 1 | 13 | 1:1 | | |
| 8 | 1 | 14 | 1:1 | | |
| 9 | 1 | 2 | 1:1 | | |
| 10 | 1 | 15 | 1:1 | | |
| 11 | 1 | 1 | 1:1 | 13.5 | 49.3 |
| 12 | 1 | 5 | 1:1 | | |
| 13 | 1 | 1 | 1:0.9 | 12.8 | |
| 14¹⁾ | 1 | 1 | 1:0.9 | 16.9 | |
| 15 | 1 | 4 | 1:0.9 | | 46.9 |
| 16 | 1 | 9 + 10 | 1:1 | | 52.9 |

¹⁾reaction not quite complete

Investigation of the viscoelastic behavior of the polyisobutenesuccinic acid ester from preparation example 11 revealed, at shear rates in the range from 10⁻³ to 10² s⁻¹ and in particular in the range from 10″3 to 101 s⁻¹ in a temperature range from 60 to 90° C., Newtonian viscoelastic behavior and viscosities in the range from 0 to 10³ Pa·s and in particular viscosities from 6 to 400 Pa·s. Above a shear rate above 10²s⁻¹, the viscosity decreases linearly to below 0 Pa·s. (see FIG. 1).

Investigation of the viscoelastic behavior of the polyisobutene succinic acid esters from preparation examples 11 and 12 revealed that the temperature profile of the viscosity depends on the molecular weight of the alcohol selected from the poly-C₂-C₄-alkylene glycols and poly-C₂-C₄-alkylene glycol mono-C₂-C₂₀-alkyl ethers. The ester from preparation example 12 (black) reveals a greater temperature dependency than the ester from preparation example 11 (grey) (see FIG. 2).

The maximum water absorption capacity of the polyisobutenesuccinic acid ester from preparation example 11 is shown in table 2 below:

TABLE 2
Water absorption capacity of preparation example 11

| Solvent temperature [° C.] | |
|---|---|
| Demin. water | Jayco solution |

| | 22 | 4 | 22 | 4 |
|---|---|---|---|---|
| Water absorption | 81 | 110 | 130 | 103 |
| [% by wt.] ²⁾ | | | | |

²⁾ Average value, based on the starting weight of the copolymer used.

### V HYDROGELS

### General Preparation Procedure.

The polyisobutenesuccinic acid ester was melted at a temperature of 70° C. and diluted with the amount of warm water and optionally surfactant given in table 3. In all cases, a clear hydrogel was formed.

### Example 1

The gel of example 21 was prepared analogously to the general preparation procedure from the polyisobutenesuccinic acid ester of preparation example 11 by dilution with water/surfactant. The hydrogel was then analyzed viscometrically. The results for example 21 are shown in FIGS. 4 and 6.

TABLE 3

| | Polyisobutene- | | | |
|---|---|---|---|---|
| | succinic acid | Water | Surfactant | AN |
| Example | ester ³⁾ | [% by wt.] ⁴⁾ | [% by wt.] ⁴⁾ | [mg KOH/g] |
| 1 | 2 | 80 | 0 | 7.5 |
| 2 | 3 | 60 | 0 | 9.5 |
| 3 | 4 | 66.6 | 0 | 5.0 |
| 4 | 5 | 66.6 | 0 | 3.6 |
| 5 | 6 | 66.6 | 0 | 8.8 |
| 6 | 7 | 40 | 0 | |
| 7 | 7 | 50 | 0 | |
| 8 | 7 | 60 | 0 | |
| 9 | 7 | 70 | 0 | |
| 10 | 8 | 40 | 0 | |
| 11 | 8 | 50 | 0 | |
| 12 | 8 | 60 | 0 | |
| 13 | 8 | 70 | 0 | |
| 14 | 8 | 14 | 6 | |
| 15 | 8 | 24 | 6 | |
| 16 | 8 | 34 | 6 | |
| 17 | 8 | 44 | 6 | |
| 18 | 10 | 36 | 0 | |
| 19 | 10 | 34 | 6 | |
| 20 | 11 | 14 | 6 | |
| 21 | 11 | 44 | 6 | |
| 22 | 11 | 54 | 6 | |
| 23 | 12 | 44 | 6 | |
| 24 | 13 | 50 | 0 | |
| 25 | 6 | 30 | 10 ⁵⁾ | |

³⁾ preparation example number
⁴⁾ based on the hydrogel
⁵⁾ mixture of 9 parts by weight of the nonionic surfactant with 1 part by weight of a customary perfume oil

1-18. (canceled)

19. The use of esters of polyisobutenesuccinic acid with an alcohol selected from poly-C₂-C₄-alkylene glycols and poly-C₂-C₄-alkylene glycol mono-C₁-C₂₀-alkyl ethers as gel former in hydrogels.

20. The use according to claim 19, where the polyisobutene radical of the ester has a number-average molecular weight in the range from 500 to 5000 daltons.

21. The use according to claim 19, where the alcohol has a number-average molecular weight in the range from 500 to 15 000 daltons.

22. The use according to claim 19, where the alcohol is selected from linear poly-C2-C₄-alkylene glycols and linear poly-C2-C4-alkylene glycol mono-C1-C20-alkyl ethers.

23. The use according to claim 19, where the alcohol is composed to at least 50 mol %, based on the total number of alkylene oxide repeat units in the alcohol, of repeat units of the formula [CH2CH2O].

24. The use according to claim 23, where the alcohol has 1 to 50 mol %, based on the total number of alkylene oxide repeat units in the alcohol, of repeat units of the formula [CH2CH(CH3)O].

25. The use according to claim 19, where the ester has on average a weight ratio of polyisobutene radical to alcohol radical in the range from 10:1 to 1:30.

26. The use according to claim 19, where the ester is obtainable by reacting polyisobutenesuccinic anhydride with an alcohol selected from poly-C2-C4-alkylene glycols and poly-C2-C4-alkylene glycol mono-C1-C22-alkyl ethers or a mixture of these alcohols.

27. The use according to claim 26, where the polyisobutenesuccinic anhydride has a saponification number in the range from 40 to 140 mg KOH/g.

28. The use according to claim 26, where the polyisobutenesuccinic anhydride comprises less than 20% by weight of polyisobutenesuccinic acid with 2 succinic acid groups per polyisobutene radical.

29. A hydrogel comprising
a. as component A, at least one ester of polyisobutenesuccinic acid with an alcohol selected from poly-C₂-C₄-alkylene glycols and poly-C₂-C₄-alkylene glycol mono-C₁-C₂₂-alkyl ethers according to claim 19 in an amount sufficient to form a hydrogel and
b. water as component B.

30. The hydrogel according to claim 29, in which the weight ratio of component A to component B is in the range from 4:1 to 1:6.

31. The hydrogel according to claim 29, where the total amount of component A and B constitutes at least 70% by weight of the hydrogel.

32. The hydrogel according to claim 29, comprising
a. 15 to 80% by weight, based on the total weight of the hydrogel, of component A and
b. 20 to 85% by weight, based on the total weight of the hydrogel, of water.

33. The hydrogel according to claim 29, comprising at least one further component C, which is selected from fragrances, surfactants, dyes, preservatives, disinfectants, complexing agents, thickeners, humectants, disintegrants, foam stabilizers and substances which dissolve lime or urine scale.

34. The hydrogel according to claim 29, which has a viscosity at 20° C. in the range from 105 to 1010 Pa·s, determined at 30° C. with a shear-stress-controlled rotary viscometer with cone/plate geometry and a shear stress range from 102 to 104 Pa.

35. A process for producing a hydrogel according to claim 29, comprising the incorporation of at least one ester of polyisobutenesuccinic acid with an alcohol selected from poly-C2-C4-alkylene glycols and poly-C2-C4-alkylene glycol mono-C1-C22-alkyl ethers in an aqueous liquid.

36. The use of a hydrogel according to claim 28 in cleaners and care compositions for the home, in cosmetics or for medical products.
